(19) United States
(12) Patent Application Publication
(10) Pub. No.: US 2012/0271130 A1
(43) Pub. Date: Oct. 25, 2012

(54) METHOD AND APPARATUS FOR DETERMINING AN OXYGEN DESATURATION EVENT

(75) Inventor: Paul Benni, Acton, MA (US)

(73) Assignee: CAS MEDICAL SYSTEMS, INC., Branford, CT (US)

(21) Appl. No.: 13/444,509

(22) Filed: Apr. 11, 2012

Related U.S. Application Data
(60) Provisional application No. 61/474,088, filed on Apr. 11, 2011.

(51) Int. Cl. A61B 5/1455; A61B 5/0205

(52) U.S. Cl. 600/324

(57) ABSTRACT

A method and apparatus for determining an index indicative of a subject's response to an oxygen desaturation condition is provided. The method includes the steps of: a) providing a NIRS tissue sensor, a pulse oximetry sensor, and a processor in communication with the NIRS tissue sensor and the pulse oximetry sensor; b) sensing the subject's tissue using the NIRS tissue sensor and producing first signals; c) sensing the subject's tissue using the pulse oximetry sensor and producing second signals; d) processing the first signals to determine a change in tissue oxygen saturation values, processing the second signals to determine a change in arterial oxygen saturation values; and e) determining the index indicative of the subject's response to the oxygen desaturation condition using the change in tissue oxygen saturation values and the change in arterial oxygen saturation values.

Applicant hereby claims priority benefits under 35 U.S.C. §119(e) of U.S. Provisional Patent Application No. 61/474,088 filed Apr. 11, 2011, the disclosure of which is herein incorporated by reference.

### BACKGROUND OF THE INVENTION

1. Technical Field

This invention relates to methods and apparatuses for non-invasively determining biological tissue oxygenation in general, and to non-invasive methods and apparatuses utilizing near infrared spectroscopy (NIRS) techniques for determining oxygen desaturation events in particular.

2. Background Information

Infants born prematurely (i.e., before 37 weeks gestation) are at increased risk for brain injuries that lead to neurodevelopmental delays (e.g., sensory-motor, cognitive, and behavioral delays). The mechanisms behind these brain injuries are poorly understood, though they are believed to be caused by a number of factors, including hypoxia (i.e., oxygen deprivation), hypoperfusion-reperfusion, inflammation, etc. The timing, extent and severity of these brain injuries influence the extent and severity of the resulting neurodevelopmental delays. Other factors, including gestational age of the infant, birth weight, intracranial pathology (e.g., intraventricular hemorrhage, peri-ventricular leukomalacia, etc.), and respiratory diseases (e.g., bronchopulmonary dysplasia, chronic lung disease, sepsis, etc.) also influence the extent and severity of the neurodevelopmental delays.

In the past, limited understanding of the brain injuries that lead to neurodevelopmental delays was due to limitations in non-invasive neuro-monitoring. The ability to assess the brains of premature infants was limited to the bedside neurologic exams and interval imaging studies (e.g., ultrasound, CT and MRI scans). These techniques had significant limitations. Most notably, brain injuries were often identified only after they occurred, and thus the mainstay of treatment was largely supportive rather than preventive. There had been no systematic way to individually identify the premature infants at high risk for a brain injury before such brain injury occurred.

### SUMMARY OF THE INVENTION

According to an aspect of the present invention, a method for non-invasively determining the ability of a subject to adapt to systemic hypoxia events is provided. The method includes the steps of: a) providing a NIRS tissue sensor, a pulse oximetry sensor, and a processor in communication with the NIRS tissue sensor and the pulse oximetry sensor; b) sensing the subject's tissue using the NIRS tissue sensor, and producing a first signal that is indicative of an oxygen saturation value of the subject's tissue, which oxygen saturation value is a composite of the microvascular (arterioles, venules, and capillaries) blood oxygen saturation within the tissue; c) sensing the tissue using the pulse oximetry sensor, and producing a second signal that is indicative of the subject's arterial oxygen saturation; d) processing the first and second signals to determine a mean tissue oxygen saturation value for each of a number of predetermined arterial oxygen saturation values; e) calculating a rate of change between the mean tissue oxygen saturation values and a rate of change for the arterial oxygen saturation values corresponding to the tissue oxygen saturation values; and f) determining an ability of the subject's body to adapt to transient or longer term systemic hypoxia, ischemia, and/or disease states.

NIRS measured tissue oxygen saturation is different than arterial oxygen saturation, with NIRS measured tissue oxygen saturation being typically lower, because tissue oxygen saturation is measured from microvascular blood (arterioles, venules, and capillaries) that is mostly venous blood by volume. Because in part tissue oxygen saturation contains venous blood, tissue oxygen saturation gives an indication of tissue oxygen utilization and demand. The arterial oxygen saturation measured by pulse oximetry is obtained from pulsatile blood, in which the pulse oximetry examines the plethysmogrograph waveform at two or more discrete light wavelengths (typically 660 and 940 nm). If the blood flow is not pulsatile, such as the case when the heart is arrested and the subject is on a cardiopulmonary bypass pump, pulse oximeters cannot function, but NIRS can still measure tissue oxygen saturation because NIRS examines non-pulsatile blood. Pulsatile components of microvascular blood are filtered out in NIRS measurements.

According to another aspect of the present invention, an apparatus for non-invasively determining the ability of a subject to adapt to transient or longer term systemic hypoxia, ischemia, and/or disease states is provided. The apparatus includes a NIRS tissue sensor, a pulse oximetry sensor, and a processor. The NIRS tissue sensor is operable to sense the subject's tissue and produce a first signal indicative of an oxygen saturation value of the subject's tissue. The pulse oximetry sensor is operable to sense tissue of the subject, and produce a second signal that is indicative of the subject's arterial oxygen saturation. The processor is in communication with the NIRS tissue sensor and the pulse oximetry sensor. The processor is adapted to process the first and second signals to determine a mean tissue oxygen saturation value for each of a number of predetermined arterial oxygen saturation values. The processor is further adapted to calculate a rate of change between the mean tissue oxygen saturation values and a rate of change for the arterial oxygen saturation values corresponding to the tissue oxygen saturation values. The processor is further adapted to determine an ability of the subject's body to adapt to transient or longer term systemic hypoxia, ischemia, and/or disease states.

According to another aspect of the present invention, a method for determining an index indicative of a subject's response to an oxygen desaturation condition is provided. The method includes the steps of: a) providing a NIRS tissue sensor, a pulse oximetry sensor, and a processor in communication with the NIRS tissue sensor and the pulse oximetry sensor; b) sensing the subject's tissue using the NIRS tissue sensor during a period of time, and producing first signals that are indicative of a tissue oxygen saturation value during the period of time; c) sensing the subject's tissue using the pulse oximetry sensor during the period of time, and producing second signals that are indicative of the subject's arterial oxygen saturation during the period of time; d) processing the first signals to determine a change in tissue oxygen saturation values over the period of time, processing the second signals to determine a change in arterial oxygen saturation values over the period of time; and e) determining the index indicative of the subject's response to the oxygen desaturation condition using the change in tissue oxygen saturation values and the change in arterial oxygen saturation values.

According to another aspect of the present invention, an apparatus for determining an index indicative of the subject's response to an oxygen desaturation condition is provided that includes a NIRS tissue sensor, a pulse oximetry sensor, and a processor. The NIRS tissue sensor is operable to sense the subject's tissue during a period of time, and produce first signals indicative of a tissue oxygen saturation value during the period of time. The pulse oximetry sensor is operable to sense the subject's tissue during the period of time, and produce second signals indicative of the subject's arterial oxygen saturation during the period of time. The processor is in communication with the NIRS tissue sensor and the pulse oximetry sensor. The processor is adapted to process the first signals to determine a change in tissue oxygen saturation values over the period of time, and to process the second signals to determine a change in arterial oxygen saturation values over the period of time. The processor is further adapted to determine the index indicative of the subject's response to an oxygen desaturation condition using the change in tissue oxygen saturation values and the change in arterial oxygen saturation values.

These and other objects, features, and advantages of the present invention method and apparatus will become apparent in light of the detailed description of the invention provided below and the accompanying drawings. The method and apparatus described below constitute a preferred embodiment of the underlying invention and do not, therefore, constitute all aspects of the invention that will or may become apparent by one of skill in the art after consideration of the invention disclosed overall herein.

### BRIEF DESCRIPTION OF THE DRAWINGS

FIG. 1 is a diagrammatic representation of the present apparatus, with a NIRS tissue sensor and a pulse oximetry sensor applied to the subject.

FIG. 2 is a diagrammatic representation of a NIRS tissue sensor placed on a subject's head.

FIG. 3 is a diagrammatic representation of a NIRS tissue sensor.

FIG. 4 is a diagrammatic representation of a pulse oximetry sensor.

FIG. 5 is a graph diagrammatically illustrating an x-axis of pulse oximetry values and a y-axis of mean SctO₂ oximetry values.

FIG. 6 is a flow chart illustrating steps according to one aspect of the present invention.

FIG. 7 is a graph diagrammatically illustrating an x-axis of time values and a y-axis of slope values.

FIG. 8 is a graph of study weight versus slope.

FIG. 9 is a graph of study gestational age versus slope.

FIG. 10 is a graph of study age versus slope.

FIG. 11 is a graph of birth weight versus slope.

FIG. 12 is a graph of birth gestational age versus slope.

### DETAILED DESCRIPTION THE INVENTION

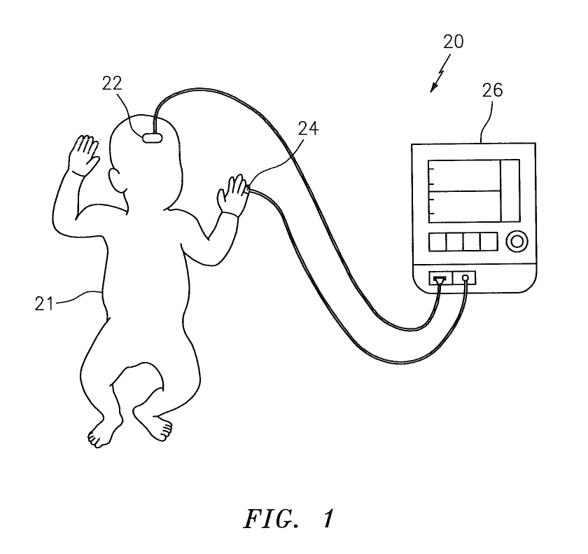

Referring to FIG. 1, the present invention includes a device 20 that utilizes both NIRS tissue oximetry and pulse oximetry to concurrently evaluate oxygen saturation levels within a subject 21, including evaluation of oxygen desaturation events as they may occur. The device includes at least one near infrared spectrophotometric (NIRS) tissue sensor 22 and at least one pulse oximetry sensor 24. The sensors 22, 24 are in communication with a processor portion 26 of the device 20 adapted to process the signals from each according to the algorithm described below.

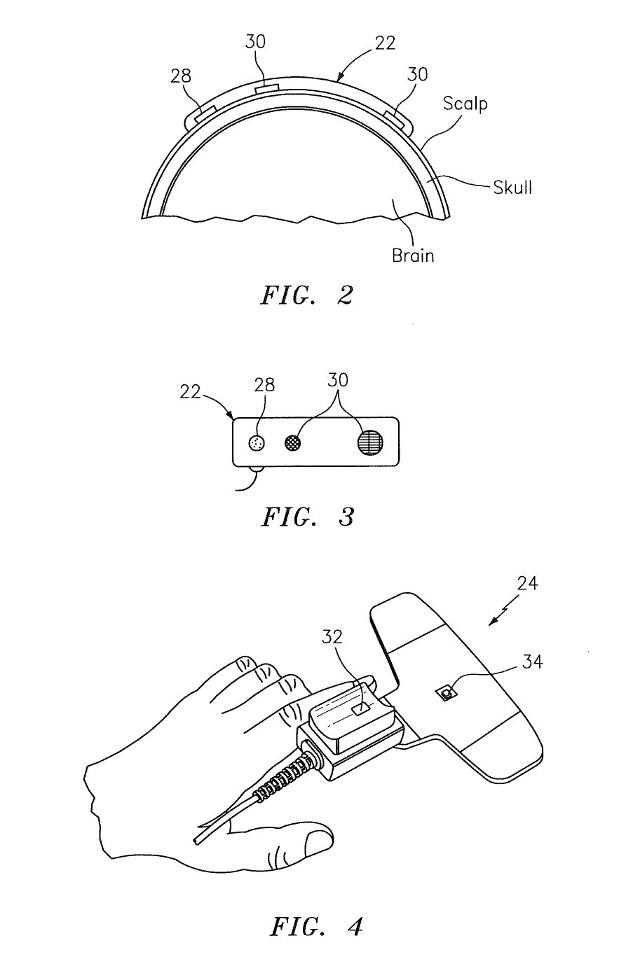

An example of an acceptable NIRS tissue sensor 22 is illustrated in FIGS. 2 and 3. The sensor 22 includes an assembly housing that is a flexible structure that can be attached directly to a subject's body, and which includes one or more light sources 28 and light detectors 30. The light source 28 may include one or more light emitters (e.g., laser diodes, LEDs, etc.) located in the sensor assembly housing, or may include a light passage (e.g., a fiber optic light guide) located in the assembly housing connected to one or more light emitters located in a processor portion of the device. The light source 28 is adapted to emit light signals of known but different wavelengths. The light detectors 30 typically include one or more photodiodes in communication with the processor portion 26 of the device via a connector cable. An example of an acceptable NIRS tissue sensor 22 is described in U.S. Patent Application Publication No. 2010/0049018, which publication is hereby incorporated by reference in its entirety.

Referring to FIG. 4, an example of a pulse oximeter sensor 24 includes a light source 32 (e.g., a pair of LEDs) and a light detector (e.g., one or more photodiodes) oppositely disposed in a housing that is attachable to a translucent part of a patient's body (e.g., fingertip, earlobe). The light source 32 typically emits red light (e.g., at about 660 nm) and infrared light (e.g., in the range of 905-940 nm). Light from the light source 32 is transmitted through the subject's tissue, and the light detector 34 detects the light that passes through the tissue. The detected light consists of two components for each bandwidth. An AC component represents the amount of pulsatile blood detected, while the DC component represents the amount of non-pulsatile blood.

The present invention is not limited to any particular type of NIRS tissue sensor 22 or pulse oximeter sensor 24.

Referring to FIG. 1, the processor portion 26 of the device 20 is adapted to control the operation of, and process signals from, the NIRS tissue sensor 22 and the pulse oximeter sensor 24. The processor portion 26 includes one or more processors, and is adapted (e.g., programmed) to selectively perform the functions as described herein. For example, the processor portion is adapted to determine the tissue oxygen saturation value for the tissue interrogated using the NIRS tissue sensor using an algorithm such as that described in U.S. Pat. No. 7,072,701, which patent is hereby incorporated by reference in its entirety. The '701 Patent describes the algorithm in the context of sensing oxygen saturation of cerebral tissue (SctO2) although the algorithm is not limited to a cerebral application. The present method and apparatus is not limited to the NIRS tissue sensing algorithm disclosed in the '701 Patent or to a cerebral tissue application, however, and other NIRS algorithms may be used alternatively. The processor portion 26 is further adapted to determine the arterial oxygen saturation value for the tissue interrogated using the pulse oximetry sensor 24. Algorithms operable to determine arterial oxygen saturation (SpO₂) from pulsatile flow are known in the art, and therefore are not described here in detail. It should be noted that the functionality of processor portion may be implemented using hardware, software, firmware, or a combination thereof A person skilled in the art would be able to program the processor portion to perform the functionality described herein without undue experimentation.

The processor portion 26 is further adapted to process the signals from the NIRS tissue sensor 22 and the pulse oximetry sensor 24 to determine an index indicative of the subject's response to an oxygen desaturation condition. To simplify the description of the present invention, the tissue oxygen saturation signals from the NIRS tissue sensor are described as being generated in an application where the cerebral tissue oxygen saturation value (SctO₂) is determined. The comparison of the SctO2 and the SpO2 measurements provide the index indicative of the subject's response to an oxygen desaturation condition; i.e., valuable information regarding the ability of the subject's body to withstand brief periods of hypoxia, ischemia, etc. that are characterized by a desaturation event. For example, the comparison of the SctO2 and the SpO2 measurements possible with the present invention can provide information relating to the presence or lack of a cerebral oxygenation reserve.

According to an embodiment of the present invention, the processor portion 26 is adapted to determine SctO₂ and SpO₂ values (e.g., simultaneously) over a given period of time based on the signals received from the NIRS tissue sensor 22 and the pulse oximetry sensor 24. In some embodiments, the algorithm organizes the determined SpO₂ values in terms of their magnitude. For example, all 100% SpO₂ values (e.g., arterial blood that is 100% oxygenated) may be grouped together; all 90% SpO₂ values are grouped together, etc. “Binning” is an example of how the values may be grouped. The specific increment used for grouping (e.g., binning) purposes (e.g., every 2%-100, 98, 96, etc.) can be varied to suit the application. Each time a SpO₂ value is generated; the SctO₂ value generated at that point in time is binned (or otherwise associated) with the SpO₂ value, and other SpO₂ values of the same magnitude. For example, all of the SctO₂ values determined at a SpO₂ value of 90% are binned together, and all of the SctO₂ values determined at a SpO₂ value of 94% are binned together, etc. The SctO₂ values for a given SpO₂ value are subsequently processed to determine a mean SctO₂ value associated with that particular SpO₂ value.

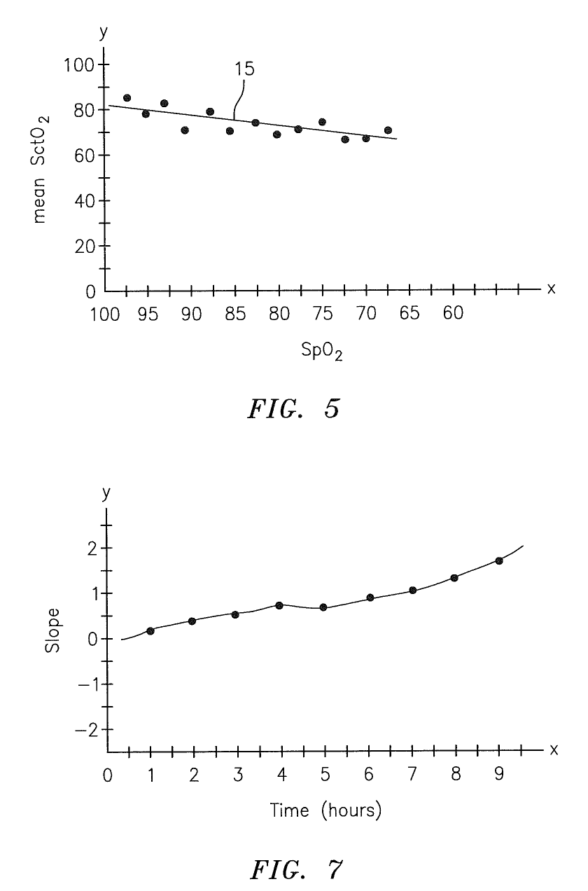

The determination of the index (e.g., by comparison of the SctO₂ and SpO₂ values) can be performed with the values organized in a variety of different forms. For example, a graphical solution can be used wherein the mean SctO₂ versus the SpO₂ values are plotted, as is shown in FIG. 5. From the graphical solution, an index value (e.g., a “slope”; referred to hereinafter as a slope, but not limited to a slope value) of the plotted SctO₂ vs. SpO₂ values can be determined Alternatively, a mathematical model (e.g., an equation) can be created that is representative of the mean SctO₂ values versus the SpO₂ values, which model includes a slope value. The range of data points used to create the graphical solution or the mathematical model can be varied to suit the application at hand. In addition, the comparison of the SctO₂ and SpO₂ values can performed, and an index (e.g., a slope value) determined, using SctO₂ and SpO₂ values organized within a database (e.g., a table) stored within the processor portion.

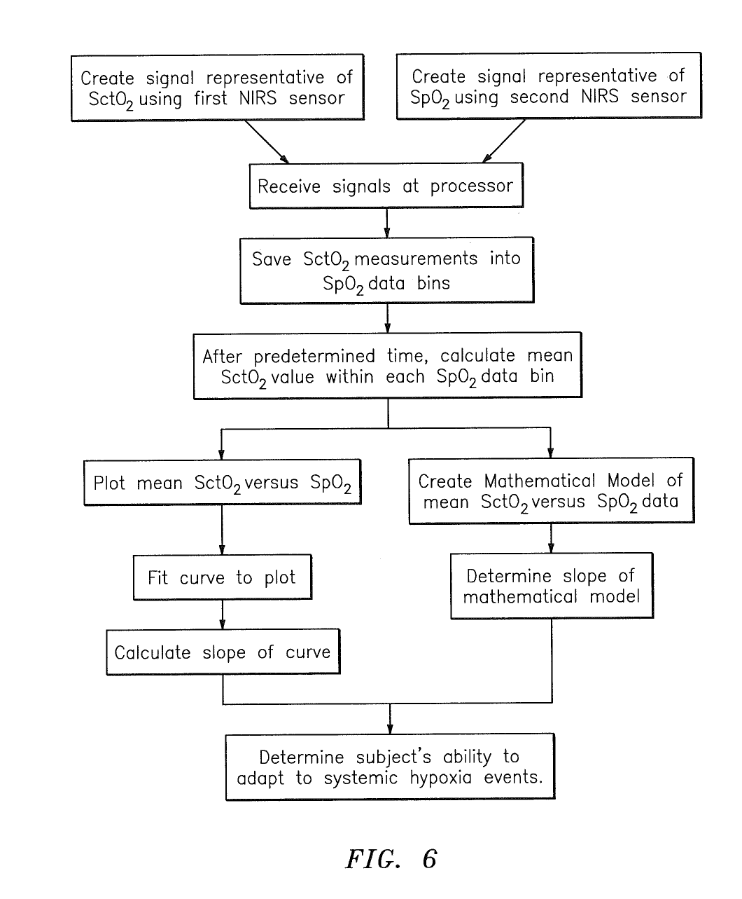

Once determined, the index (e.g., slope value) of the data (e.g., the difference in SctO₂ over the difference in SpO₂ for a given period of time) can be used to evaluate an oxygen desaturation event within the subject, and subsequently provide clinical data that can be used to evaluate the condition of the subject based on the oxygen desaturation event. FIG. 6 illustrates embodiments of the algorithm in block diagram form. The slope provides information on the ability of the subject's body to withstand and adapt to brief episodes of hypoxia, ischemia, etc., that are characterized by a desaturation event; i.e., an indicator of how well the subject's brain is being protected. For example, a slope of one (1) indicates that the change in SpO₂ versus the change in SctO₂ (i.e., change in the SaO₂−SctO₂ difference) remains constant during the time period wherein oxygen desaturation event(s) occurred, which is an indication that the body's physiological response did not adapt or adjust to the arterial oxygen desaturation event(s). In instances where a body's physiological response does not adapt or adjust to an arterial oxygen desaturation event(s), it is possible there is no cerebral oxygen reserve. A slope of less than one (<1) indicates that the change in SpO₂ versus the change in SctO₂ (i.e. change in the SaO₂−SctO₂ difference) is decreasing, with lower SpO₂ values occurring during the arterial oxygen desaturation event(s). The lower SpO₂ values are an indicator that the body's physiological response adapted and adjusted to the oxygen desaturation event(s). As a result, a slope of less than one (<1) indicates that there is cerebral oxygen reserve, whereas increasing brain oxygenation reserve is indicated by smaller slope values. For example, if the SctO₂ value does not appreciably change over the given time period, and the SpO₂ experiences a transient decrease (e.g., from 100% to 80%), then the slope value will approach zero, which indicates the body adaptation “methods” completely protected the brain from the desaturation event. Examples of the aforesaid adaptation methods (i.e., how the subject's body protects the subject's brain from hypoxia and ischemia) include cerebral autoregulation, carotid body response, increased heart rate, stroke volume, and/or cardiac output, etc. A slope of greater than one (>1) indicates that there may be additional physiological issues with the brain in terms of oxygenation; e.g., a blockage of blood flow within the arteries passing blood to the brain. A range of SpO₂ values facilitates accurate calculation of the slope and the extent to which a brain oxygenation reserve is present. Accurate determination of the slope and/or the magnitude of brain oxygenation reserve is more difficult where SpO₂ is consistently the same value or deviates within only a very small range of values.

### CLINICAL APPLICATION EXAMPLES

An example of a clinical application of the present method and apparatus involves monitoring short term or transient hypoxia and/or ischemia oxygen desaturation events in neonates suffering from Respiratory Distress Syndrome (RDS). RDS is a breathing disorder that affects newborns. RDS rarely occurs in full-term infants, however. The disorder is more common in premature infants born about 6 weeks or more before their full term due date. RDS may occur more often in premature infants due to an insufficient amount of surfactant being produced within the infants' lungs. The surfactant is a liquid that coats the inside of the lungs and helps to keep the infant's lungs open post birth, which in turn facilitates the infant's breathing in air. In the absence of a sufficient amount of surfactant, the infant's lungs can collapse to some degree and make it more difficult for the infant to breathe. In some instances of RDS, the infant may not be able to breathe in enough oxygen to support the body's organs and organ damage can occur unless corrective action is taken. Using the present invention, the SpO₂ and SctO₂ values of the subject can be monitored via the pulse oximetry sensor and the NIRS tissue sensor, respectively, to determine if a desaturation event occurs during each episode where the baby struggles to breathe. The information relating to the SctO2 and SpO2 values (e.g., the index) determined by the present invention, provides clinical information to the clinician regarding any desaturation event that may have occurred and also clinical information regarding how the infant's body compensation methods are protecting the brain, and/or an indication of the infant's brain oxygen reserve.

Another clinical application of the present invention relates to determining an index indicative of a subject's response to oxygen desaturation conditions that may occur in subjects adapting to high altitude, or to subjects having congenital heart disease. The present invention can be used to determine the aforesaid index and subsequently evaluate how the subject's body's compensation methods are protecting the brain and give an indication of brain oxygenation reserve during longer term oxygen desaturation events. For example, within the present invention the SctO₂/SpO₂ slope can provide clinical information relating to a subject's adaptation to high altitude, or relating to the effects of congenital heart disease on a subject, on relating to how a subject's body compensation methods are protecting the brain, which in turn can give an indication of brain oxygenation reserve during longer term oxygen desaturation events. The human body can adapt to higher altitudes through immediate and long-term acclimatization. At high altitude, in the short term, the lack of oxygen is sensed by the carotid bodies, which causes an increase in the breathing rate (hyperventilation). However, hyperventilation also causes the adverse effect of respiratory alkalosis, inhibiting the respiratory center from enhancing the respiratory rate as much as would be required. An inability to increase the breathing rate can be caused by inadequate carotid body response or pulmonary or renal disease. In addition, at high altitude, the heart beats faster; the stroke volume is slightly decreased; and non-essential body functions are suppressed, food digestion efficiency declines (as the body suppresses the digestive system in favor of increasing its cardiopulmonary reserves). But full acclimatization requires days or even weeks. As indicated in the “Effects of High Altitude on Humans”, published in Wikipedia, the human body gradually compensates for the respiratory alkalosis by renal excretion of bicarbonate, allowing adequate respiration to provide oxygen without risking alkalosis. It typically takes about 4 days for this to occur at any given altitude and is greatly enhanced by acetazolamide. Eventually, the body has lower lactate production (because reduced glucose breakdown decreases the amount of lactate formed), decreased plasma volume, increased hematocrit (polycythemia), increased RBC mass, a higher concentration of capillaries in skeletal muscle tissue, increased myoglobin, increased mitochondria, increased aerobic enzyme concentration, increase in 2,3-BPG, hypoxic pulmonary vasoconstriction, and right ventricular hypertrophy. Adaptation of infants to congenital heart disease is similar, with increased red blood cell numbers and hemoglobin to increase hematocrit, and increased capillaries in tissue (angiogenesis). Full hematological adaptation to high altitude is achieved when the increase in number of red blood cells reaches a plateau and stops. After that period, the subject below extreme altitude (e.g., 5,500 meters˜18,000 ft) is able to perform his activities as if he were at sea level.

In some embodiments, the processor portion 26 is further adapted to evaluate the slope value (e.g., the difference in SctO₂ over the difference in SpO₂ for a given period of time) over an extended period of time. For example, if the slope is determined periodically for a given increment of time (e.g., Δt), the slope for each increment of time can be compared against other increments of time (e.g., plot the incremental slope values versus time) to evaluate trending, etc. Trending may indicate whether a subject's brain oxygen reserve is changing. As indicated above, the range of the slope values under consideration for evaluation can be chosen based on the application at hand. FIG. 7 illustrates a plot of incremental slope values versus time.

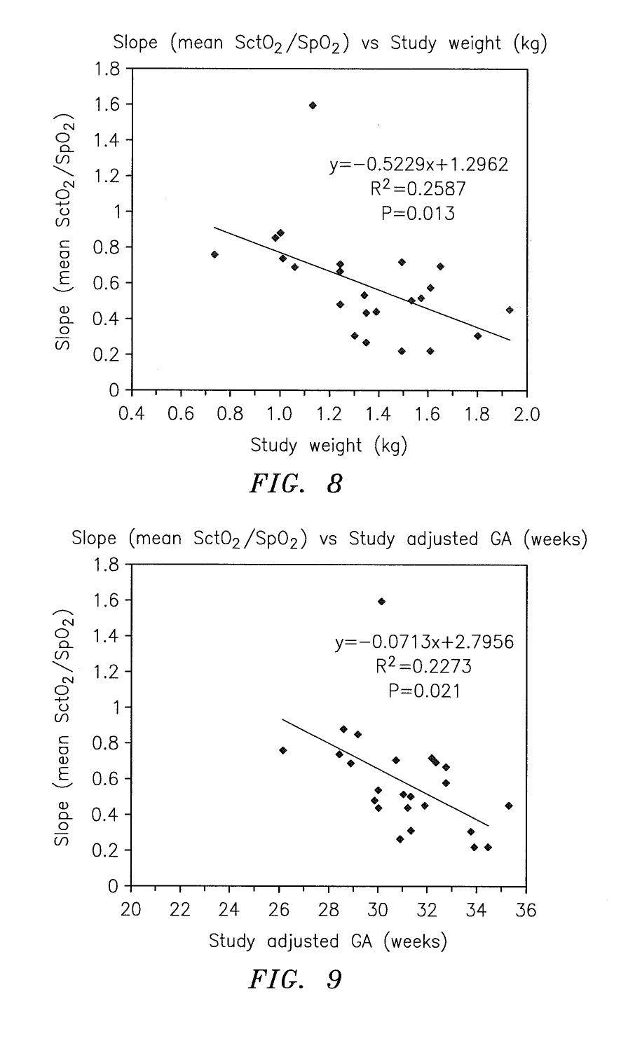

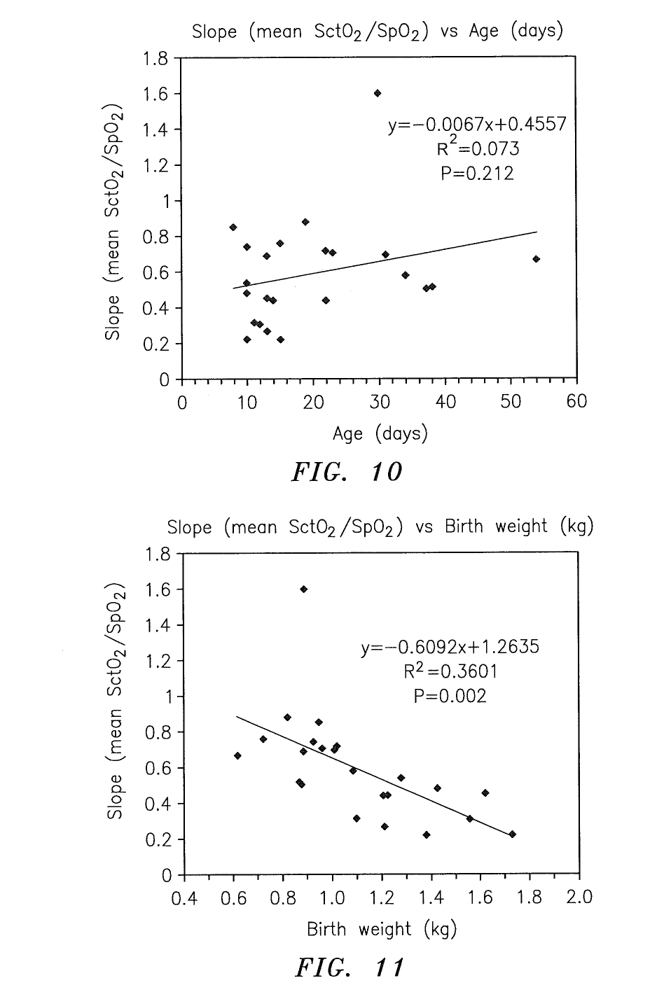

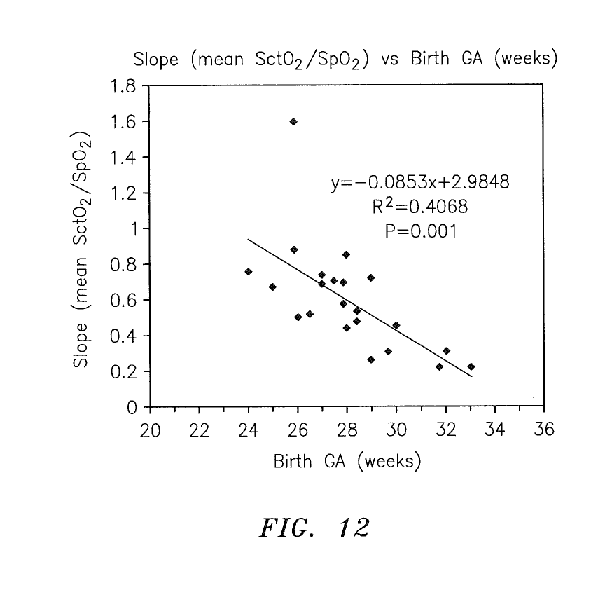

In some embodiments, the processor is further adapted to produce analytical data by comparing index values (e.g., slope values determined in the manner described above) versus a characteristic of the subject; e.g., the study weight of the subjects involved (FIG. 8), the study gestational age (GA) of the subject (FIG. 9), the number of days old the subject was at the time of analysis (i.e., study age in days; see FIG. 10), the subject's birth weight (FIG. 11), the birth gestational age (FIG. 12), etc. The comparative data produced in these embodiments is indicative of the ability of the subject's body to protect its brain during desaturation events, and how that ability relates to the subject's weight, gestational age, etc. In this way, analytical data be developed (e.g., in the form of growth-type charts) to show normal (e.g., 50^{th} percentile) and full percentile range (0 to 100 percentile) on the subject's brain oxygenation reserve as indicated by the slope as it pertains to body growth and improvement of immature physiological processes due to immaturity from pre-team birth. FIGS. 8-12 include examples of such data in graphical form, including linear modeling of the data which can also be modeled by mathematical equation. These graphs represent an example of data produced by an embodiment of the present method and are provided to illustrate the present invention, but are not limiting examples of the invention.

Although this invention has been shown and described with respect to the detailed embodiments thereof, it will be understood by those skilled in the art that various changes in form and detail thereof may be made without departing from the spirit and the scope of the invention.

What is claimed is:

1. A method for determining an index indicative of a subject's response to an oxygen desaturation condition, the method comprising the steps of:
providing a NIRS tissue sensor, a pulse oximetry sensor, and a processor in communication with the NIRS tissue sensor and the pulse oximetry sensor;
sensing the subject's tissue using the NIRS tissue sensor during a period of time, and producing first signals that are indicative of a tissue oxygen saturation value during the period of time;
sensing the subject's tissue using the pulse oximetry sensor during the period of time, and producing second signals that are indicative of the subject's arterial oxygen saturation during the period of time;
processing the first signals to determine a change in tissue oxygen saturation values over the period of time, processing the second signals to determine a change in arterial oxygen saturation values over the period of time; and
determining the index indicative of the subject's response to the oxygen desaturation condition using the change in tissue oxygen saturation values and the change in arterial oxygen saturation values.

2. The method of claim 1, wherein the determining step includes the step of comparing the change in tissue oxygen saturation values and the change in arterial oxygen saturation values over the period of time.

3. The method of claim 2, wherein the step of comparing includes determining a ratio of the change in tissue oxygen saturation values over the change in arterial oxygen saturation values, over the period of time.

4. The method of claim 2, further comprising the step of providing clinical data based on the comparison value.

5. The method of claim 2, wherein the step of determining an index indicative of the subject's response to the oxygen desaturation condition includes determining a relative rate of change between the tissue oxygen saturation values and the arterial oxygen saturation values.

6. The method of claim 2, wherein the step of determining the index utilizes one or more of a graph, a mathematical equation, and a database.

7. The method of claim 2, further comprising the step of producing clinical data based on a comparison of the index and a characteristic of the subject.

8. The method of claim 7, wherein the characteristic of the subject is one or more of the study weight of the subject, the study gestational age of the subject, the number of days old the subject was at the time of the sensing, the subject's birth weight, and the birth gestational age.

9. The method of claim 1, including the step of determining a trend of the oxygen desaturation condition.

10. A method for non-invasively determining the ability of a subject to adapt to brief systemic hypoxia and/or ischemia events, or a longer term adaptation to abnormal physiology, the method comprising the steps of:
providing a NIRS tissue sensor, a pulse oximetry sensor, and a processor in communication with the NIRS tissue sensor and the pulse oximetry sensor;
sensing the subject's tissue using the NIRS tissue sensor, and producing a first signal that is indicative of an oxygen saturation value of the subject's tissue;
sensing the tissue using the pulse oximetry sensor, and producing a second signal that is indicative of the subject's arterial oxygen saturation;
processing the first and second signals to determine a mean tissue oxygen saturation value for each of a number of predetermined arterial oxygen saturation values;
calculating a rate of change between the mean tissue oxygen saturation values and a rate of change for the arterial oxygen saturation values corresponding to the tissue oxygen saturation values; and
determining an ability of the subject's body to adapt to systemic hypoxia events.

11. An apparatus for determining an index indicative of the subject's response to an oxygen desaturation condition, comprising:
a NIRS tissue sensor operable to sense the subject's tissue during a period of time, and produce first signals indicative of a tissue oxygen saturation value during the period of time;
a pulse oximetry sensor operable to sense the subject's tissue during the period of time, and produce second signals indicative of the subject's arterial oxygen saturation during the period of time; and
a processor in communication with the NIRS tissue sensor and the pulse oximetry sensor, which processor is adapted to process the first signals to determine a change in tissue oxygen saturation values over the period of time, and to process the second signals to determine a change in arterial oxygen saturation values over the period of time, and to determine the index indicative of the subject's response to an oxygen desaturation condition using the change in tissue oxygen saturation values and the change in arterial oxygen saturation values.

12. The apparatus of claim 11, wherein the processor is further adapted to compare the change in tissue oxygen saturation values and the change in arterial oxygen saturation values over the period of time.

13. The apparatus of claim 12, wherein the processor is adapted to determine a ratio of the change in tissue oxygen saturation values over the change in arterial oxygen saturation values, over the period of time.

14. The apparatus of claim 11, wherein the processor is further adapted to produce clinical data based on the index.

15. The apparatus of claim 11, wherein the processor is adapted to determine a relative rate of change between the tissue oxygen saturation values and the arterial oxygen saturation values.

16. The apparatus of claim 11, wherein the processor is adapted to determine the index using one or more of a graph, a mathematical equation, and a database.

17. The apparatus of claim 11, wherein the processor is adapted to produce clinical data based on a comparison of the index and a characteristic of the subject.

18. The apparatus of claim 17, wherein the characteristic of the subject is one or more of the study weight of the subject, the study gestational age of the subject, the number of days old the subject was at the time of the sensing, the subject's birth weight, and the birth gestational age.

19. The apparatus of claim 11, wherein the processor is adapted to determine a trend of the oxygen desaturation condition.

20. An apparatus for non-invasively determining the ability of a subject to adapt to brief systemic hypoxia and/or ischemia event, or an longer term adaptation to abnormal physiology, comprising:
a NIRS tissue sensor operable to sense the subject's tissue and produce a first signal indicative of an oxygen saturation value of the subject's tissue;
a pulse oximetry sensor operable to sense tissue of the subject, and produce a second signal that is indicative of the subject's arterial oxygen saturation; and
a processor in communication with the NIRS tissue sensor and the pulse oximetry sensor, which processor is adapted to process the first and second signals to determine a mean tissue oxygen saturation value for each of a number of predetermined arterial oxygen saturation values, and to calculate a rate of change between the mean tissue oxygen saturation values and a rate of change for the arterial oxygen saturation values corresponding to the tissue oxygen saturation values, and to determine an ability of the subject's body to adapt to brief systemic hypoxia and/or ischemia event, or an longer term adaptation to abnormal physiology.
